(19) United States
(12) Patent Application Publication
(10) Pub. No.: US 2021/0277383 A1
(43) Pub. Date: Sep. 9, 2021

(54) PURIFICATION AND ISOLATION OF SYNTHETIC OLIGONUCLEOTIDES USING HYDROPHILIC-INTERACTION LIQUID CHROMATOGRAPHY

(71) Applicant: Waters Technologies Corporation, Milford, MA (US)

(72) Inventors: Jo-Ann Jablonski, Middleboro, MA (US); Catalin Doneanu, Franklin, MA (US)

(73) Assignee: Waters Technologies Corporation, Milford, MA (US)

(21) Appl. No.: 17/192,429

(22) Filed: Mar. 4, 2021

Related U.S. Application Data
(60) Provisional application No. 62/986,421, filed on Mar. 6, 2020.

(51) Int. Cl. C12N 15/10; G01N 30/72; G01N 30/74; G01N 30/14

(52) U.S. Cl. CPC C12N 15/101; G01N 30/7233; G01N 2030/027; G01N 30/14; G01N 30/74

(57) ABSTRACT

Methods of purifying targeted oligonucleotides within a reaction mixture using hydrophilic interaction liquid chromatography (HILIC) is disclosed. One of the methods in accordance with the present disclosure includes screening the targeted oligonucleotides within the reaction mixture with HILIC to create an initial reaction mixture profile; determining an elution percentage for the targeted oligonucleotides; focusing a HILIC elution gradient around the elution percentage of the targeted oligonucleotides; and purifying the targeted oligonucleotides with HILIC using the focused elution gradient at room temperature. Some embodiments can utilize mass triggering for fraction collection of the targeted oligonucleotides. Some embodiments can utilize UV triggering when the mass falls outside of the mass range of the MS detector.

### CROSS-REFERENCE TO RELATED APPLICATIONS

This application claims priority to U.S. Provisional Patent Application No. 62/986,421 filed on Mar. 6, 2020, entitled “Purification and Isolation of Synthetic Oligonucleotides Using Hydrophilic-Interaction Liquid Chromatography”. The contents of which is incorporated herein by reference in its entirety.

### FIELD OF THE TECHNOLOGY

The present disclosure relates to methods for the purification of synthetic oligonucleotides. More specifically, the present disclosure relates to methods that provide improved purification of synthetically made oligonucleotides. In general, methods of the present disclosure utilize Hydrophilic-Interaction Liquid Chromatography (HILIC), and some embodiments also include a mass-targeted isolation of a desired synthetically-made oligonucleotide to achieve the improved purification.

### BACKGROUND

Synthetic oligonucleotides are short nucleic acid chains that can act in a sequence specific manner to control gene expression. And, as a result, synthetic oligonucleotides are of great interest to the biopharmaceutical and personal medicine markets.

To obtain the desired oligonucleotide, the building blocks (e.g., A, C, G, U, etc.) are sequentially coupled to the growing oligonucleotide chain in a desired order. Upon completion of the assembly, the product is released from a solid phase support into solution (i.e., a reaction mixture) and collected.

This step-wise chemical process used to prepare the desired oligonucleotide introduces many complex by-products, adducts, and deleted/truncated sequences into the reaction mixture. Typically, targeted or desired oligonucleotides contained in the reaction mixture require isolation and purification before they can be used experimentally or for a desired medical/pharmaceutical use. Common techniques employed for the purification of this crude reaction mixture include gel electrophoresis, ion-exchange chromatography, and reverse phase liquid chromatography. These techniques have limitations, however. For example, gel electrophoresis purification techniques typically result in the loss of mass as it is nearly impossible to recover all product from a gel-slice. Ion-exchange chromatography involves the use of buffers that typically contain non-volatile salts, which increase impurities and the process involves unavoidable losses of product.

Purification of the crude reaction mixture by reverse phase liquid chromatography (RPLC) is challenging. First, there is a narrow separation space (e.g., usually less than 10% change over a full separation gradient), which reduces the probability of successfully separating the product and impurity peaks, a critical factor which impacts the feasibility of isolating pure targeted molecules. In addition to the narrow separation space, common mobile phase additives for RPLC possess unpleasant odors and their toxicity make them difficult and costly to handle. Elevated temperature requirements of RPLC add to the difficulty of the purification process. In general, RPLC is conducted within the temperature range of 60 to 90 degrees Celsius. Due to problems stemming from temperature distribution, temperature gradients occur not only from the entrance to the exit of the column, but also across its width (e.g., diameter). These temperature gradients effect the quality of isolation and thus degrade the possible purification. And effective temperature control on large preparative columns is not easily realized. Since reversed phase chromatography for oligonucleotides usually requires a temperature range of 60 to 90 degrees Celsius and temperature control for larger, preparative columns is not easily realized, performing preparative chromatography at room temperature is preferred.

### SUMMARY

A solution is needed to overcome the challenges associated with the common techniques for purifying synthetically made oligonucleotides (e.g., RPLC, ion-exchange chromatography, and gel electrophoresis) and to also improve the purification process in general (e.g., lower cost, higher efficiency, reduced purification time, greater purity, greater amount). In some examples, utilizing mass-directed purification for targeted products having mass-to-charge ratios falling within the mass range of the instrument can reduce the ambiguity that can accompany separations performed with ultraviolet (UV) detection only. In some examples, using shorter columns for preparative oligonucleotide purification can improve productivity and reduce cost. In some embodiments, isolating and purifying the targeted oligonucleotides at room temperature provides improved and/or repeatable results. By using these solutions, and in some examples in conjunction with other solutions, the challenges associated with the common techniques for analysis/purification of oligonucleotides are reduced or eliminated using HILIC methodologies.

In one aspect, the technology is directed to a method of separating a reaction mixture comprising synthetic oligonucleotides using hydrophilic interaction liquid chromatography (HILIC). The method includes (1) isolating targeted oligonucleotides in the reaction mixture with HILIC; and (2) purifying the isolated targeted oligonucleotides with a mass-directed technique, wherein the isolated targeted oligonucleotides have a mass to charge ratio falling within a specified mass range.

In another aspect, the technology is directed to a method of separating a reaction mixture comprising synthetic oligonucleotides using hydrophilic interaction liquid chromatography (HILIC). The method includes (1) isolating targeted oligonucleotides in the reaction mixture with HILIC; and (2) purifying the isolated targeted oligonucleotides to a final purity of greater than or equal to 95.0%.

In another aspect, the technology is directed to a method of purifying targeted oligonucleotides within a reaction mixture using hydrophilic interaction liquid chromatography (HILIC). The method includes (1) screening the targeted oligonucleotides within the reaction mixture with HILIC to create an initial reaction mixture profile; (2) determining an elution percentage for the targeted oligonucleotides; (3) focusing a HILIC elution gradient around the elution percentage of the targeted oligonucleotides; and (4) purifying the targeted oligonucleotides with HILIC using the focused elution gradient at room temperature.

In another aspect, the technology is directed to a method of separating a reaction mixture comprising synthetic oligonucleotides. The method includes (1) isolating targeted oligonucleotides in the reaction mixture with a column having a length between 50 to 100 mm; and (2) purifying the isolated targeted oligonucleotides with a mass-directed technique, wherein the isolated targeted oligonucleotides have a mass to charge ratio falling within a specified mass range.

Embodiments of the above aspects can include one or more of the following features. For example, in some embodiments, purifying the isolated targeted oligonucleotides comprises purifying to a final purity of greater than or equal to 95.0%. In some embodiments, purifying the oligonucleotides comprises purifying to a final purity of greater than or equal to 99.0%. In some embodiments, using the mass-directed technique comprises using a mass spectrometer (MS) having detection capabilities within the specified mass range. In some embodiments, using the mass-directed technique comprises using a system having both a mass spectrometer and an ultraviolet (UV) detector. In some embodiments, isolation of the oligonucleotides is performed at room temperature and room temperature can range from about 20° C. to about 25° C. In some embodiments, isolating with HILIC comprises eluting with a mobile phase having a pH greater than about 5. In some embodiments, an additional step is performed after purification. The additional step includes performing an orthogonal method technique on the targeted oligonucleotides for fraction analysis.

### BRIEF DESCRIPTION OF THE DRAWINGS

The technology will be more fully understood from the following detailed description taken in conjunction with the accompanying drawings, in which:

FIG. 1 is a flow chart illustrating a method in accordance with the present disclosure.

FIG. 2A is a chromatogram of a 16-mer oligonucleotide purification by HILIC.

FIG. 2B is a chromatogram of a 30-mer oligonucleotide purification by HILIC.

FIG. 2C is a chromatogram of a 57-mer oligonucleotide purification by HILIC.

FIG. 3A is an UV chromatogram for a 16-mer oligonucleotide fraction.

FIG. 3B is a TIC chromatogram for a 16-mer oligonucleotide fraction.

FIG. 4A is an UV chromatogram for a 30-mer oligonucleotide fraction.

FIG. 4B is a TIC chromatogram for a 30-mer oligonucleotide fraction.

FIG. 5A, FIG. 5B, and FIG. 5C provide data processing results for a 57-mer oligonucleotide fraction.

### DETAILED DESCRIPTION

The synthesis of oligonucleotides can create complex by-products, adducts, and truncated sequences. A reaction mixture containing the waste products as well as the targeted oligonucleotides requires isolation and purification before use. That is, in order to use the synthetic oligonucleotides in a product (e.g., biopharmaceutical or personalized medicine etc.), the crude reaction mixture needs to be separated to isolate and purify the oligonucleotides (e.g., to remove the waste products from the targeted oligonucleotides).

RPLC, as discussed, provides a narrow separation space. In some examples, the separation space can be a 3 to 5% organic gradient change. The narrow separation can create difficulties when pulling away targeted oligonucleotides and also for maintaining a high purity. In some examples, unwanted products can coelute with the desired product, e.g., the targeted oligonucleotides. The unwanted products negatively impact the purity of the targeted oligonucleotides. In addition to the narrow separation space, RPLC has other downsides/challenges. For example, buffers, modifiers, and additives of RPLC (e.g., trimethylamine (TEA) and 1,1,1,3,3,3-hexafluoro-2-propanol (HFIP)) can be toxic, which makes handling costly and risky. In addition, HFIP is expensive irrespective of the handling issues. These costs can make RPLC cost-prohibitive, requiring other solutions. In general, oligonucleotide separations using RPLC require high temperatures. For example, RPLC can commonly require column temperatures of 60 to 90 degrees Celsius for the separation to occur. RPLC also requires long columns, such as 150 mm to 250 mm. The long columns can increase the size of the fraction volume, which can increase the fraction drying time and the ability to process samples in a timely manner. The length of time for RPLC can impact the process and the costs associated with RPLC. In addition, RPLC is typically used with ultraviolet (UV) detection only for isolation steps. UV detection results in non-specific analysis. That is, UV absorption is not that specific—mass analysis (MS detector) is much more specific, and can provide more targeted isolation capability.

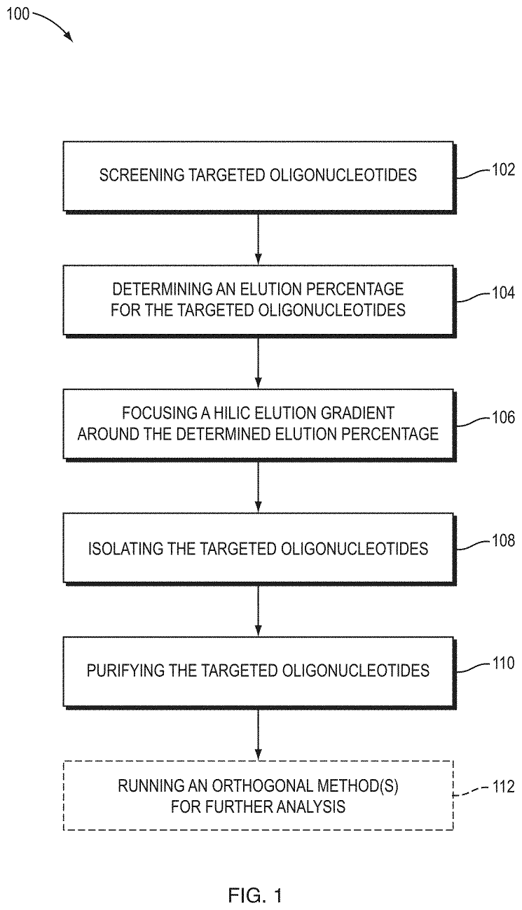

HILIC alleviates the challenges associated with reversed phase chromatography for the isolation and purification of oligonucleotides. FIG. 1 is a flow chart illustrating a method 100 in accordance with the present disclosure. Method 100 includes optional steps as indicated by dashed lines in FIG. 1. Method 100 for HILIC chromatography with mass-directed purification includes screening targeted oligonucleotides (102) in the crude reaction mixture, determining an elution percentage for the targeted oligonucleotides (104), focusing a HILIC elution gradient around the determined elution percentage (106), isolating the targeted oligonucleotides (108), purifying the targeted oligonucleotides (110), and optionally running an orthogonal method(s) for further analysis, such as fraction analysis (112).

In some examples, screening targeted oligonucleotides (102) can be referred to as a scouting run. The scouting run can help determine an appropriate gradient for the isolation of a targeted oligonucleotide. With HILIC separation of oligonucleotides, the narrow separation space problem experienced with RPLC is virtually eliminated. Whereas a 3 to 5% total screening separation space (e.g., gradient from 7 to 12%) is available in a RPLC technique, a 5 to 50% or more (i.e., a 45% total) broad space is available for screening targeted oligonucleotides (102) in the methods of the present disclosure. A screening gradient with a broad eluent range provides the present methods with a crude reaction mixture profile that is wide enough to determine an elution percentage for the targeted oligonucleotides (104). The retention time of the oligonucleotide product, along with the system and column volumes and the gradient slope are used to develop a focused gradient. For isolating the targeted oligonucleotides (108), focusing the gradient around the oligonucleotide elution percentage (106) improves the resolution between the product and its closely eluting contaminant peaks and increases the likelihood of obtaining pure product. Screening gradients can range in size. For example, screening gradients can be from 5-50% or 5-95% strong solvent for the initial analysis. The screening gradients can enable oligonucleotide isolations to be subsequently performed using focused gradients with shallow slopes (e.g., 0.2-0.4% per column volume) and a range around the target product elution percentage. The range around the target product elution percentage can vary. For example, the range can be 8% with 5% below the target and 3% above the target to ensure isolation of the targeted oligonucleotides.

Reversed phase oligonucleotide separations are usually performed at elevated temperature (60 to 90 degrees Celsius). Although temperature control is often employed in analytical chromatography to improve separations, effective temperature control on larger preparative columns is not so easily realized. Heat jackets placed around the column are not always effective. The separation occurs at the temperature of the incoming solvent in an equilibrated column, thereby requiring the solvent to be heated before it enters the column. Temperature gradients generally occur in more than one dimension (e.g., not only through the length, but also width of column). The result can be a column that is not in equilibrium. HILIC separations for oligonucleotides at room temperature eliminate the inherent complications associated with heating preparative columns. In some examples, room temperature extends from about 20 to about 25 degrees Celsius (° C.).

Mobile phase buffers containing TEA and HFIP are commonly used for reversed phase methods employing mass spectrometry with electrospray ionization. Although HFIP increases sensitivity in the mass analysis of oligonucleotides, as reported in the literature, HFIP is toxic and its use can be cost-prohibitive.

The buffers, modifiers, and additives for HILIC can be common (e.g., ammonium acetate), less toxic than mobile phase buffers such as TEA and HFIP, easy-to-handle, and reasonably priced (not cost-prohibitive). The mobile phases of the present disclosure can contain 20 millimolar (mM) ammonium acetate with the pH adjusted to 5.5. In some examples, the pH is greater than 5. The pH can be adjusted according to process conditions, including the targeted oligonucleotide. For example, different oligonucleotide sequences may require different ammonium acetate and/or pH adjustments. Some advantages of ammonium acetate include: compatible with mass spectrometers, evaporates easily during lyophilization, and is of reasonable cost. Ammonium acetate can be used for positive-negative switching in mass detection, and oligonucleotides are mostly detected in negative ionization mode.

For isolating the targeted oligonucleotides (108) with HILIC, short columns can be used. In some examples, short columns include 2.1×50 millimeter (mm) or 2.1×100 mm. Benefits of short columns include less time and smaller volume of fraction which can be mostly organic solvent that can dry more quickly than water. These benefits positively impact the process and costs. In addition, the present disclosure is not restricted to 2.1 mm columns. The process can be scaled to larger diameter columns, including 7.8, 10, 19, 30, 50 mm (and, potentially, even larger diameter columns) depending on the amount of product to be purified. These example diameters for columns are not exhaustive, and other diameters can be used as well. The benefits for using shorter columns even with a larger diameter are still realized. And, in some examples, longer columns could be used as well. In some examples, the determination of column size/dimension can be based on, at least in part, on the crude oligonucleotide reaction mixture and the desired product.

Although UV is universally used for oligonucleotide detection at both the analytical and preparative scales, MS-directed purification reduces the ambiguity associated with UV detection alone. Most systems with mass detection are also configured with a UV/PDA detector, and both the UV and MS chromatograms are collected at the same time. The dual detection is useful for those instances where the compound mass falls outside the mass range of the mass detector or for those compounds that have limited ionization potential. Targeted collection of known masses increases the likelihood of isolating the correct product more efficiently. Because oligonucleotides are susceptible to multiple mass charges, masses which fall within the m/z range of the mass spectrometer are sometimes available for targeting even through the molecular mass of the full length is higher than the upper mass limit of the MS. Software programs which calculate the multiple charges of the oligonucleotide assist in providing suggested targets for MS-directed purification. For example, a 16-mer using mass-directed purification with multiple charge states specified as fraction triggers.

Results

Shorter columns than those which would be required for reversed phase separations were used for the isolation of three oligonucleotides, a 16-mer on a 2.1×50 mm Torus DIOL column, and a 30-mer and a 57-mer on a 2.1×100 mm Torus DIOL column, both with 1.7 μm particle size. Short columns save time and processing resources. All columns are commercially available from Waters Technologies Corporation (Milford, Mass., USA).

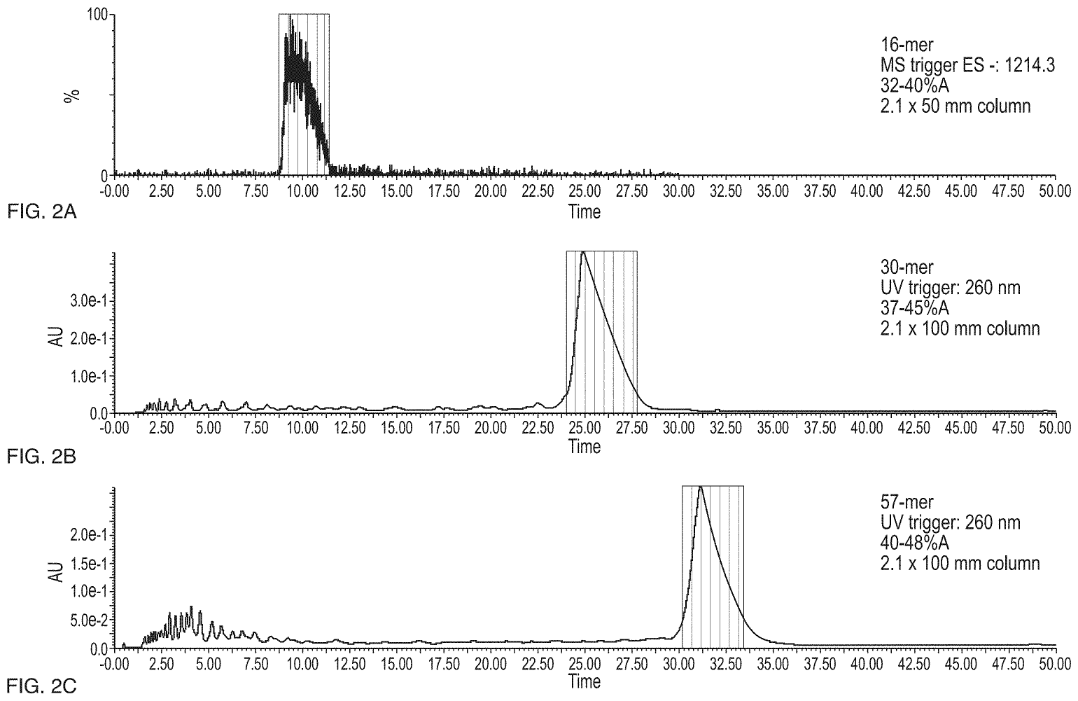

FIG. 2A provides a chromatogram of a 16-mer oligonucleotide purification by HILIC in accordance with the technology of the present disclosure. In this purification of a 16-mer oligonucleotide, the following mobile phase was used: mobile phase A included 95 water/5 acetonitrile with 20 mM ammonium acetate, pH 5.5 and mobile phase B included 5 water/95 acetonitrile with 20 mM ammonium acetate, pH 5.5. As the oligonucleotide is a short oligonucleotide with a molecular weight of approximately 4800 g/mol, it has a mass to charge ratio that falls within the detection capabilities of the MS device configured in the system of the present disclosure. Other mass spectrometers on different systems may have broader mass ranges. From a screening run, the MS trigger for isolation of the 16-mer was determined to be 1214.3. The isolation is focused around the elution percentage of the target oligonucleotide, and the mass of the target oligonucleotide is used to trigger fraction collection. FIG. 2A demonstrates that the purification was mass-directed with the isolations conditions for the 16-mer focused around the elution percentage of the target oligonucleotide.

FIG. 2B provides a chromatogram of a 30-mer oligonucleotide purification by HILIC in accordance with the technology of the present disclosure. In this purification of a 30-mer oligonucleotide, the following mobile phase was used: mobile phase A included 95 water/5 acetonitrile with 20 mM ammonium acetate, pH 5.5 and mobile phase B included 5 water/95 acetonitrile with 20 mM ammonium acetate, pH 5.5. This oligonucleotide has a higher molecular weight (e.g., approximately 9200 g/mol) as compared to the 16-mer. The 30-mer does not have mass charge states falling within the mass range of the mass detector configured in the system of the present disclosure. As a result, the UV wavelength giving maximum compound absorbance was used as the UV trigger. In this example, the UV trigger was determined to be 260 nm. Similar to FIG. 2A, FIG. 2B demonstrates that the purification was UV-directed with the isolation conditions focused around the elution percentage of the targeted oligonucleotide.

FIG. 2C provides a chromatogram of a 57-mer oligonucleotide purification by HILIC in accordance with the technology of the present disclosure. In this purification of a 57-mer oligonucleotide, the following mobile phase was used: mobile phase A included 95 water/5 acetonitrile with 20 mM ammonium acetate, pH 5.5 and mobile phase B included 5 water/95 acetonitrile with 20 mM ammonium acetate, pH 5.5. This oligonucleotide also has a higher molecular weight (e.g., approximately 17,500 g/mol) as compared to the 16-mer. The 57-mer also does not have a mass to charge ratio falling within the detection capabilities of a MS device. As a result, a UV detector is used and a UV trigger is identified from a screening run. In this example, the UV trigger was determined to be 260 nm. FIG. 2C demonstrates that the purification was UV-directed with the isolation conditions focused around the elution percentage of the targeted oligonucleotide. Focusing was around the elution percentage of the compound and the MS or UV wavelength is used for detection and fraction triggering. Many compounds absorb at a single wavelength, whereas one (or a fewer number of compounds) will have the specified mass.

In these examples, oligonucleotides of average length were successfully isolated using HILIC chromatography on 50 and 100 mm Torus DIOL columns using reasonably-priced mobile phase additive and UV and/or MS-directed purification. The isolation was done at room temperature with stable and less toxic mobile phase additive. The product fractions were high in organic solvent content and were easily evaporated for post-purification analysis with an orthogonal method. The final product quality was excellent as detailed in Table 1.

TABLE 1
HILIC Purification (Oligonucleotide purity table)

| OLIGONUCLEOTIDE | CRUDE PURITY | FINAL PURITY |
|---|---|---|
| 16-mer | 57.0% | 95.2% |
| 30-mer | 87.9% | 99.5% |
| 57-mer | 75.3% | 99.5% |

In some examples, purifying the isolated targeted oligonucleotides includes purifying to a final purity of greater than or equal to 60%, 65%, 70, 75%, 80.0%, 85.0%, 90.0%, 95.0%, 99%, 99.5%, or higher.

Several different HILIC columns were part of the experimental studies, and any of them might be acceptable for other oligonucleotides, depending on the properties of the molecule. Some of the columns included BEH HILIC, BEH Amide, Atlantis HILIC Silica, and Torus DIOL, all in either 50 mm or 100 mm lengths with 2.1 mm ID. The Atlantis HILIC Silica column was packed with 3 μm particles, while the other columns were packed with 1.7 μm particles. The isolation of small amounts of oligonucleotides was successful using the H-Class System configured with the Waters Fraction Manager-Analytical (WFMA) and the DIOL columns. All equipment is commercially available from Waters Technologies Corporation (Milford, Mass., USA). The process can be scaled to include larger columns and systems, including columns such as 7.8, 10, 19, 30, 50 mm (and, potentially, even larger diameter columns).

Particle size of the column packings may be selected based on predicted column use. Column packings with larger particle sizes may also be used. For examples, 1.7 μm, 3 μm, 5 μm, and 10 μm can be used. Larger particles may potentially be used for large scale purifications.

Fraction Analysis of the Targeted Oligonucleotide—Orthogonal Results

The product fractions were high in organic solvent content and were evaporated for post-purification analysis with an orthogonal method such as ion-pair RP separation with mass analysis.

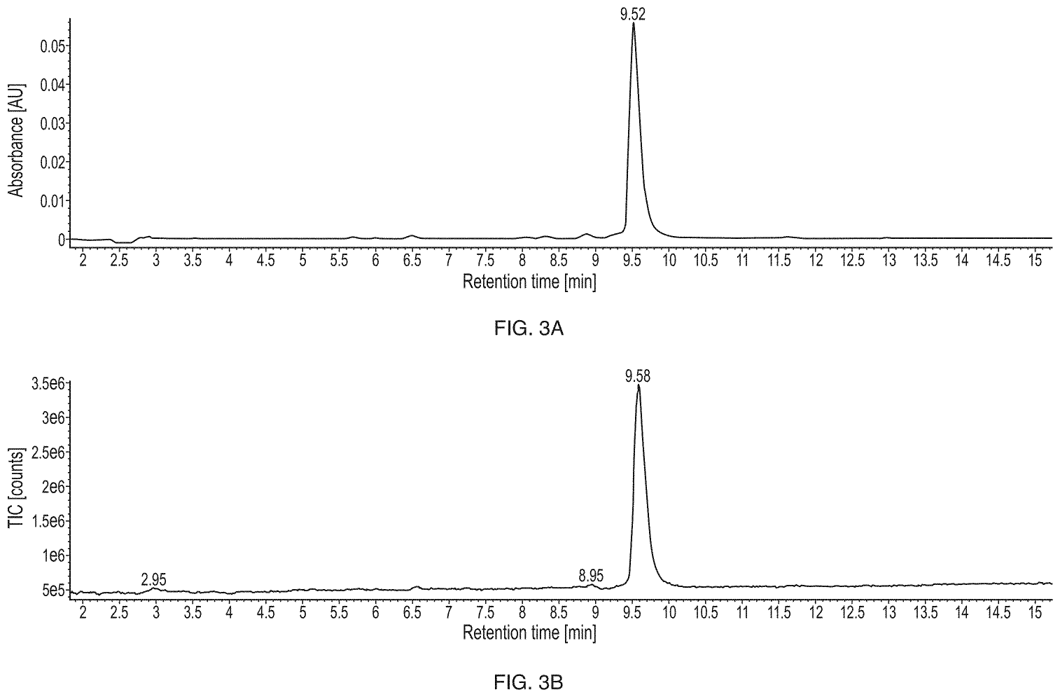

FIG. 3A is an UV chromatogram for a 16-mer oligonucleotide fraction. The orthogonal analysis used an ion-pair separation on a 2.1×50 mm OST column, and the temperature was maintained at 60 degrees Celsius. For the mobile phase, there are two solvents: solvent A and solvent B. Solvent A was 15 mM TEA and 400 mM HFIP in water. Solvent B was 15 mM TEA and 400 mM HFIP in methanol. The flow rate was 0.2 mL/min. In this example, the ion pairing separation was monitored at 260 nm.

FIG. 3B is a TIC chromatogram for a 16-mer oligonucleotide fraction. The orthogonal analysis used an ion-pair separation on a 2.1×50 mm OST column, and the temperature was maintained at 60 degrees Celsius. For the mobile phase, there are two solvents: solvent A and solvent B. Solvent A was 15 mM TEA and 400 mM HFIP in water. Solvent B was 15 mM TEA and 400 mM HFIP in methanol. The flow rate was 0.2 mL/min. In this example, the UV trigger was determined to be 260 nm. The conditions described for FIG. 3A and FIG. 3B are for the same experiment performed with UV detection (FIG. 3A) and MS detection (FIG. 3 B). A comparison of the results shown in FIGS. 3A and 3B illustrates the high purity of the HILIC based purification methods of this disclosure.

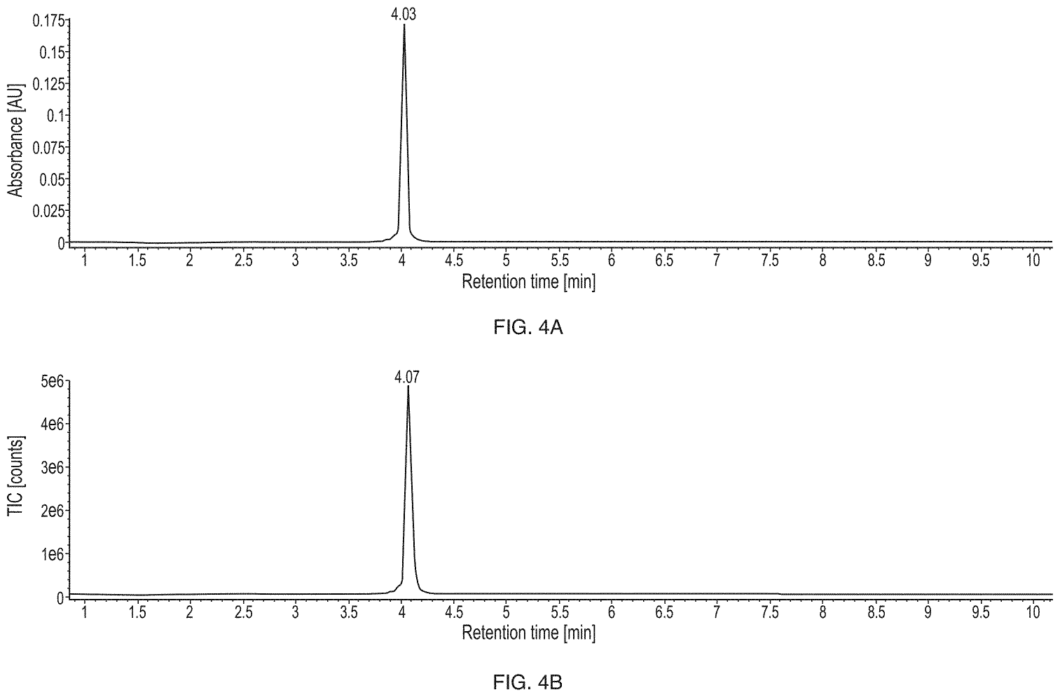

FIG. 4A is an UV chromatogram for a 30-mer oligonucleotide fraction. The orthogonal analysis used an ion-pair separation on a 2.1×50 mm OST column, and the temperature was maintained at 60 degrees Celsius. For the mobile phase, there are two solvents: solvent A and solvent B. Solvent A was 7 mM TEA and 80 mM HFIP in water. Solvent B was 3.5 mM TEA and 40 mM HFIP in 50% methanol. The flow rate was 0.3 mL/min. In this example, the ion pairing separation was monitored at 260 nm.

FIG. 4B is a TIC chromatogram for a 30-mer oligonucleotide fraction. The orthogonal analysis used an ion-pair separation on a 2.1×50 mm OST column, and the temperature was maintained at 60 degrees Celsius. For the mobile phase, there are two solvents: solvent A and solvent B. Solvent A was 7 mM TEA and 80 mM HFIP in water. Solvent B was 3.5 mM TEA and 40 mM HFIP in 50% methanol. The flow rate was 0.3 mL/min. In this example, the UV trigger was determined to be 260 nm. A comparison of the results shown in FIGS. 4A and 4B illustrates the high purity of the HILIC based purification methods of this disclosure.

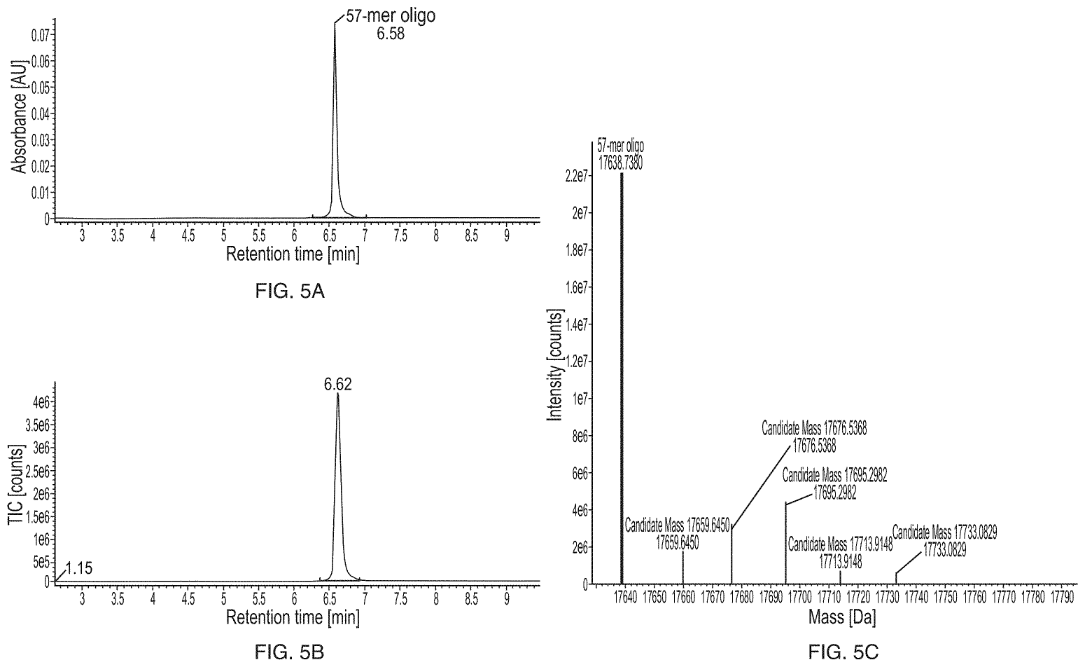

FIG. 5A, FIG. 5B, and FIG. 5C provide data processing results for a 57-mer oligonucleotide fraction. The data processing result in UNIFI had a 24 ppm mass accuracy error. The orthogonal analysis used an ion-pair separation on a 2.1×50 mm OST column, and the temperature was maintained at 60 degrees Celsius. The mobile phase contained two solvents: Solvent A was 7 mM TEA and 80 mM HFIP in water, while Solvent B was 3.5 mM TEA and 40 mM HFIP in 50% methanol. The flow rate was 0.3 mL/min. The ion pairing separation was monitored at 260 nm. The chromatograms on the left side portion are from a reverse phase ion pairing analysis (left side, top) and a TIC analysis (left side, bottom). The chromatograms on the left side are from a reversed phase ion pairing analysis with UV analysis at 260 nm (top) and mass analysis (total ion chromatogram, bottom). These results together with data processing using UNIFI (right side portion) illustrate the high purity of the disclosed purification process.

Alternatives

There are a number of alternatives methods and embodiments available for use in the present disclosure. While the above methods have generally been discussed with respect to using a mobile phase including 20 mM ammonium acetate with the pH adjusted to 5.5, other buffers, amounts of buffer, and pH values are possible. Further, while the above examples demonstrate preparative chromatography at a small scale, it is expected that the methods described herein can be translated and scaled for the isolation of larger amounts of target compound. These and other alternatives or embodiments are possible and practicable by those of ordinary skill in the art.

1. A method of separating a reaction mixture comprising synthetic oligonucleotides using hydrophilic interaction liquid chromatography (HILIC), the method comprising:
isolating targeted oligonucleotides in the reaction mixture with HILIC; and
purifying the isolated targeted oligonucleotides with a mass-directed technique, wherein the isolated targeted oligonucleotides have a mass to charge ratio falling within a specified mass range.

2. The method of claim 1, wherein purifying the isolated targeted oligonucleotides comprises purifying to a final purity of greater than or equal to 95.0%.

3. The method of claim 1, wherein using the mass-directed technique comprises using a mass spectrometer having detection capabilities within the specified mass range.

4. The method of claim 1, wherein using the mass-directed technique comprises using a system having both a mass spectrometer and an ultraviolet (UV) detector.

5. A method of separating a reaction mixture comprising synthetic oligonucleotides using hydrophilic interaction liquid chromatography (HILIC), the method comprising:
isolating targeted oligonucleotides in the reaction mixture with HILIC; and
purifying the isolated targeted oligonucleotides to a final purity of greater than or equal to 95.0%.

6. The method of claim 5, further comprising performing the isolating of the oligonucleotides at room temperature and wherein room temperature ranges from about 20° C. to about 25° C.

7. The method of claim 5, wherein isolating with HILIC comprises eluting with a mobile phase having a pH greater than about 5.

8. A method of purifying targeted oligonucleotides within a reaction mixture using hydrophilic interaction liquid chromatography (HILIC), the method comprising:
screening the targeted oligonucleotides within the reaction mixture with HILIC to create an initial reaction mixture profile;
determining an elution percentage for the targeted oligonucleotides;
focusing a HILIC elution gradient around the elution percentage of the targeted oligonucleotides; and
purifying the targeted oligonucleotides with HILIC using the focused elution gradient at room temperature.

9. The method of claim 8, wherein purifying the oligonucleotides comprises purifying to a final purity of greater than or equal to 95.0%.

10. The method of claim 8, wherein purifying the oligonucleotides comprises purifying to a final purity of greater than or equal to 99.0%.

11. The method of claim 8, further comprising performing an orthogonal method technique on the targeted oligonucleotides for fraction analysis.

12-15. (canceled)
